(19) United States
(12) Patent Application Publication
(10) Pub. No.: US 2015/0322423 A1
(43) Pub. Date: Nov. 12, 2015

(54) PROTEIN ALIGNMENT METHOD

(71) Applicant: JAPAN INTERNATIONAL RESEARCH CENTER FOR AGRICULTURAL SCIENCES, Tsukuba-shi, Ibaraki (JP)

(72) Inventors: Akihiko Kosugi, Tsukuba-shi (JP); Yutaka Mori, Tsukuba-shi (JP); Kiyohiko Igarashi, Tsukuba-shi (JP)

(73) Assignee: JAPAN INTERNATIONAL RESEARCH CENTER FOR AGRICULTURAL SCIENCES, Tsukuba-shi, Ibaraki (JP)

(21) Appl. No.: 14/387,347

(22) PCT Filed: Mar. 25, 2013
(86) PCT No.: PCT/JP2013/058609

§ 371 Date: Sep. 23, 2014

(30) Foreign Application Priority Data
Mar. 24, 2012 (JP) 2012-068516

(51) Int. Cl. C12N 11/14

(52) U.S. Cl. CPC C12N 11/14

(57) ABSTRACT

A method for aligning proteins is provided. The method for aligning the proteins (20 and 32) is capable of aligning proteins (20 and 32) on the substrate (10) comprising carbon. The proteins (20 and 32) are used as structures comprising a cellulose binding domain (22) of the cellulosome and a cohesin domain (21), and a protein (30) to be bonded to the protein (20) is used as a protein (32) having a dockerin domain (31). The cohesin domain (21) can be used as an amino-acid sequence for a cohesin domain (21) derived from various microorganisms, and a functional protein can be used as a protein structure having a microbial-species-specific dockerin domain. The protein (20) can be arrayed regularly along a graphite hexagonal-plate-shaped crystal, and any protein (32) having this dockerin domain (20) can be bonded and arranged as an active protein (32) on the graphite (10).

### TECHNICAL FIELD

This invention relates to a protein alignment method. Particularly, this invention relates to a protein alignment method, which can be used as a basic applied technology for nanotechnology and the like.

### BACKGROUND ART

Various researches were conducted to clarify reaction that occurs on the solid-liquid interface between cellulose and enzyme. Thermophilic anaerobic bacteria (hereafter referred to as “Clostridium thermocellum), which is a subject to study by the inventors, decompose cellulose most quickly on the earth. It is known that cellulosome, which is an enzyme complex, plays a key to decompose cellulose. It has been found by the molecular genetic research conducted so far that cellulosome has a structure completely different from that of fungal enzyme. However, the mechanism of cellulose decomposition is not clear, and it is expected that the mechanism will be clarified soon.

In the Non-Patent Literature 1, it is reported that the enzyme moves on the crystalline cellulose due to the reaction between the crystalline cellulose, which is fixed on the hydrophobic surface of the graphite, and the enzyme and that monomolecular movement of fungal cellulase can be visualized by an atomic force microscope.

When protein is adsorbed nonspecifically to the graphite surface, a structural change occurs and it loses its function of protein. No active protein, which is fixed on a carbon material such as graphite and does not lose its function, has been confirmed.

### CITATION LIST

### Non-Patent Literature

Non-Patent Literature 1: Kiyohiko Igarashi, Anu Koivula, Masahisa Wada, Satoshi Kimura, Merja Penttila and Masahiko Samejima, “High Speed Atomic Force Microscopy Visualizes Processive Movemnet of Trichoderma reesi Cellobiohydrolase I on Crystalline Cellulose”, Journal of Biological Chemistry, December 25, Vol. 284, No. 52, pp. 36186-36190, 2009
Non-Patent Literature 2: N. Kodera, D. Yamamoto, R. Ishikawa, and T. Ando, “Video imaging of walking myosin V by high-speed atomic force microscopy”, Nature 468, 72-76, 2010.
Non-Patent Literature 3: Demain A L, Newcomb M, Wu J H., Microbiol, “Cellulase, clostridia, and ethanol”, Microbiol. Mol. Biol. Rev., Vol. 69, No. 1., pp. 124-154, March 2005
Non-Patent Literature 4: Fierobe H P, Mingardon F, Mechaly A, Belaich A, Rincon M T, Pages S, Lamed R, Tardif C, Belaich J P, Bayer E A, “Action of designer cellulosomes on homogeneous versus complex substrates: controlled incorporation of three distinct enzymes into a defined trifunctional scaffoldin”, J Biol Chem., 280(16), pp 16325-16334, Epub 2005 Feb. 10

### SUMMARY OF INVENTION

### Technical Problem

Based on the conventional knowledge, it is expected that when a scaffold protein comprising an enzyme complex (cellulosome), which helps decompose cellulose, is adsorbed nonspecifically to graphite, it loses its function as a scaffold protein.

This invention aims to provide a method to array active protein on the surface of a carbon material and the like.

### Solution to Problem

When the inventors added the partial structure of cellulosome, which was prepared by a gene recombinant technology, to the graphite and observed with an atomic force microscope, it was found that multiple scaffold proteins arrayed regularly along the graphite hexagonal-plate-shaped crystals and the enzyme subunits of cellulosome were bonded to the scaffold proteins arrayed on the graphite causing their self-organization. This finding led to this invention.

This invention supplies a method for aligning proteins, comprising; arraying proteins on a carbon substrate.

In the above method, the cellulose binding domain of cellulosome, cohesin domain and dockerin domain are used to align proteins. Preferably, a structure consisting of the cellulose binding domain of cellulosome and the cohesin domain is formed, and protein containing the dockerin domain is bonded to the cohesin domain. It is acceptable to use any cohesin domain, which has an amino acid sequence of cohesin domain derived from various microorganisms. It is also acceptable to bind the dockerin domain, which microbial species have, to the functional protein of enzymes and the like to arrange several types of protein on a substrate by using the cohesin-dockerin interaction specific to the microbial species. For the substrate, it is preferred to use mineral materials made of carbon or to use graphite.

By using the protein alignment method described in any of the above, a composite substrate, in which proteins are arrayed on a substrate made of carbon, can be obtained.

### Advantageous Effect of Invention

According to this invention, proteins can be arrayed on a substrate made of carbon. For example, by using the cohesin domain of scaffold protein of cellulosome and the dockerin domain, which the enzyme subunit of cellulosome has, proteins can be arrayed on a substrate without losing their function.

### BRIEF DESCRIPTION OF DRAWINGS

FIG. 1 shows a pattern diagram of the composite related to the embodiment of this invention.

FIG. 2 shows a pattern diagram of the partial scaffold protein related to the embodiment of this invention.

FIG. 3 shows a pattern diagram of the method for aligning scaffold proteins on a substrate surface, related to the embodiment of this invention.

FIG. 4 shows a pattern diagram of the composite related to an alternative of the protein alignment method in this invention.

FIG. 5 shows an image of a high-speed atomic force microscope; CipA, which is a scaffold protein, is arrayed on the HOPG sample disk.

FIG. 6 shows an image of a high-speed atomic force microscope; mCipA is arrayed on the HOPG sample disk.

FIG. 7 shows the bond of the scaffold protein and CelA, (a) shows a pattern diagram and (b) shows its image of a high-speed atomic force microscope.

FIG. 8 shows the bond of the scaffold protein and CelS, (a) shows a pattern diagram and (b) shows its image of a high-speed atomic force microscope.

### REFERENCE SIGN LIST

1, 1A: Composite
10: Substrate
10A: Surface of substrate
20: Scaffold protein
20A: Partial scaffold protein
21: Cohesin domain
22: Cellulose binding domain
23: Cohesin domain of different microbial species
30: Protein
31: Dockerin domain
32: Functional protein containing dockerin
40: Functional protein of different microbial species
41: Dockerin domain of different microbial species
42: Functional protein containing dockerin of different microbial species
CelA: endo-type cellulase
CelS: Exo-type cellulase

### DESCRIPTION OF EMBODIMENTS

This invention is described below in detail while referring to the drawings, embodiments and specific examples.

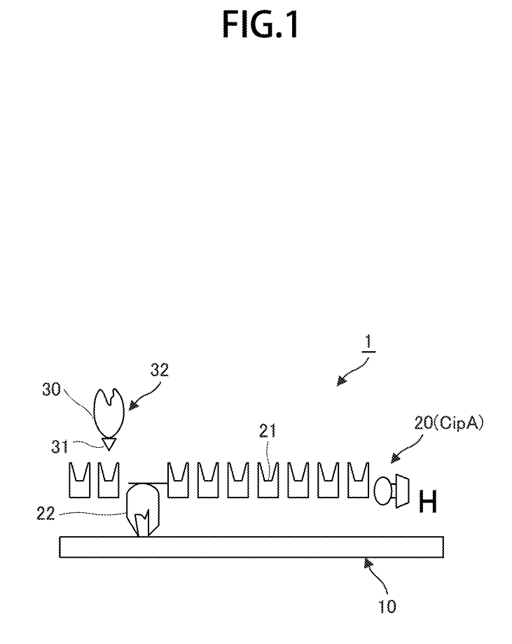

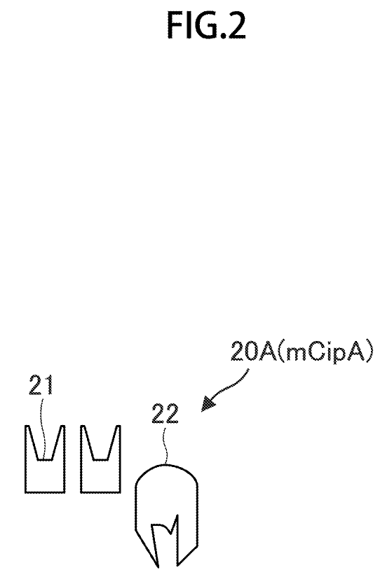

FIG. 1 shows a pattern diagram of the outlined composite 1 related to the embodiments of this invention, and FIG. 2 shows a pattern diagram of the outlined partial scaffold protein 20A related to the embodiments of this invention.

As shown by the figures, the composite 1 consists of the substrate 10 and protein containing the scaffold protein 20 such as cellulosome arrayed on the substrate 10. Namely, the protein consists of the scaffold protein 20 such as cellulosome, and the functional protein 32 containing the dockerin bonded to this scaffold protein 20. The protein structure consisting of the protein 30 and dockerin domain 31 is called the functional protein 32 containing dockerin.

Cellulosome has a scaffolding protein, which is the scaffold protein 20 for retaining cellulase and the scaffolding protein is retained on the cell surface, and cellulase is bonded to the scaffolding protein. It is reported that the scaffolding protein has the cellulase binding domain 22 and the cohesin domain 21 and that the cellulase constructing the cellulosome has the dockerin domain 31, which is bonded to the cohesin domain 21 in noncovalent bond such as hydrogen bonding (See the Non-Patent Literature 3).

Carbon materials and the like made of only carbon atoms can be used for the substrate 10. Graphite, carbon nanotube and the like can be used as carbon materials. Graphite is a native element made of carbon and has hexagonal-plate-shaped crystals. Graphite is a layered material shaped like a turtle shell. Within the in-plane of each layer, carbons are bonded with each other by strong covalent bond, but the layers are bonded with each other by weak van der Waals binding. In embodiments, graphite is used for the substrate 10 and explanation is made accordingly.

For the substrate 10, not only the graphite but any material, which has affinity with the cellulose binding domain 22, within a molecule, shown in FIG. 1 and with the cellulose binding domain 22 shown in FIG. 2 and described later, can be used. For example, the material can include cellulose.

In the composite 1 in this Embodiment, as shown in FIG. 1, the scaffold protein 20 or the partial scaffold protein 20A (see FIG. 2) is arrayed and bonded on the surface of substrate 10 through the cellulose binding domain 22.

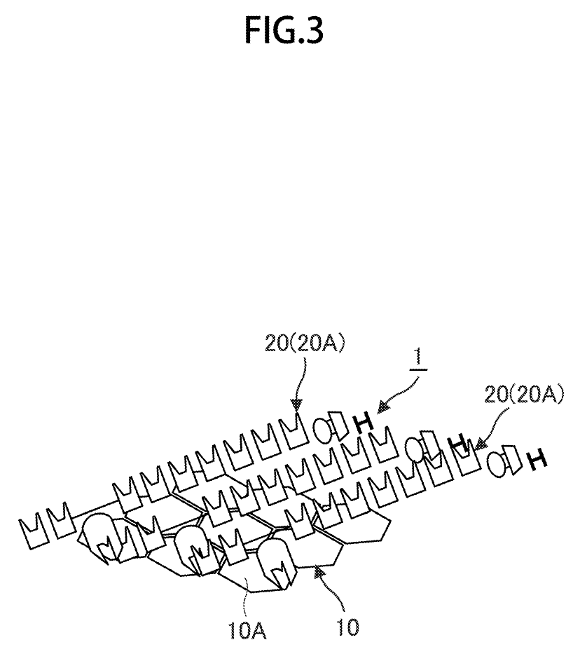

FIG. 3 shows an outline pattern diagram illustrating the method for aligning the scaffold protein 20 on the surface 10A of the substrate 10 related to the embodiments of this invention.

As shown in FIG. 3, the composite 1 is produced by adding the water solution containing multiple scaffold proteins 20 or partial scaffold proteins 20A on the surface of the face 10A, which is constructed by carbon atoms arranged like a hexagon net. The scaffold protein 20 or the partial scaffold protein 20A is arrayed on the face 10A of the substrate 10. In addition, the functional protein 32 containing the dockerin can be bonded to the scaffold protein 20 or partial scaffold protein 20A as shown in FIG. 1.

The dockerin domain 31 of the functional protein 32 containing the dockerin is bonded to the cohesin domain 21, and so the functional proteins 32 can be arrayed on the substrate 10 through a structure consisting of the cellulose binding domain 22 of cellulosome and the cohesin domain 21. For the functional protein 32 containing dockerin, a protein containing the dockerin domain 31 as its original property can be used.

It is a common property that enzyme subunits constructing the cellulosome have the dockerin domain 31 consisting of about 24 amino acids. It has been known that the dockerin domain 31 binds with the hydrophobic cohesin domain 21, which exists in the scaffold protein, in a high binding constant. The interaction between these two dockerin domain 31 and cohesin domain 21 is the basic mechanism forming the cellulosome (see Non-Patent Literature 3), and this protein-protein interaction has a relatively high binding constant.

It has been known that the above cohesin-dockerin interaction is species-specific in microorganism. Namely, the dockerin domain 31 of Clostridium thermocellum binds with the cohesin domain 21 of Clostridium thermocellum. The dockerin domain of Clostridium cellulolyticum binds with the cohesin domain of Clostridium cellulolyticum. Therefore, production of artificial cellulosome by using these properties is reported (see the Non-Patent Literature 4).

For example, as described later, the cohesin domain 21 of Clostridium thermocellum can be change into the amino acid sequence of cohesin domain of Clostridium cellulovorans, which produces cellulosome similarly. In that case, the functional protein containing a dockerin can be used as a dockerin domain derived from the same microorganism as the cohesin domain. Namely, functional protein containing the dockerin domain of Clostridium cellulovorans can be bonded to the above cohesin domain. By using this interaction of microbial-species-specific cohesin-dockerin domains, two or more, several types of proteins can be arranged and arrayed without losing their original function.

(Example of Scaffold Protein)

A specific example of scaffold protein 20 is described. As scaffold protein 20, an enzyme cloned from the one contained in cellulosome (enzyme complex) can be used. This enzyme has as its original property the dockerin domain 31, which binds with the cohesin domain 21 that constructs the scaffold protein 20.

One example of the scaffold protein 20 is CipA, and this CipA (196 kDa) has nine cohesin domains 21 and one cellulose binding domain 22 as shown in FIG. 1. The cellulose binding domain 22 is arranged between the second cohesin domain 21 and the third cohesin domain 21. For these cohesin domains 21, the scaffold protein derived from microorganism, for example, Clostridium thermocellum can be used.

Instead of the scaffold protein 20, the scaffold protein 20A, which is a part of it, can be used. The partial scaffold protein 20A shown in FIG. 2 is mCipA (its molecular weight is 53 kDa), and this mCipA is a partial structure of CipA from the N terminal of CipA to the cellulose binding domain 22. mCipA has the cellulose binding domain 22 and two cohesin domains 21 as shown in FIG. 2. These scaffold protein 20 and partial scaffold protein 20A are produced by a recombination technology.

(Dockerin Domain)

In addition, similarly the above dockerin domain derived from microorganism can be used as the dockerin domain 31, which has affinity with the cohesin domain 21 of the scaffold protein 20. For the functional protein 32 containing the dockerin having the dockerin domain 31, any of enzyme, antibody and labeled compound can be used.

For the dockerin domain 31 in this Embodiment, the one derived from microorganism same as the cohesin domain 21 can be used. For example, for the scaffold protein 20 and the partial scaffold protein 20A, which is a part of it, the following proteins derived from microorganism can be used.

Clostridium cellulovorans
Clostridium cellulolyticum
Clostridium josui
Clostridium acetobutylicum
Clostridium cellobioparum
Clostridium papyrosolvens
Ruminococcus albus
Ruminococcus flavefaciens
Acetivibrio cellulolyticus
Bacteroides cellulosolvens
Butyrivibrio fibrisolvens

(One Example of Method for Producing Composite)

One example of the method for arraying the scaffold protein 20 and the functional protein 32 containing the dockerin, which binds with this scaffold protein, is described below.

Firstly, water solution containing the scaffold protein 20 is added on the substrate 10 made of graphite. Then multiple scaffold proteins 20 are arrayed on the graphite surface.

Subsequently, as the functional protein 32 containing the dockerin, the water solution containing the enzyme 30 is added on the substrate 10. Then, the dockerin domain 31 of the enzyme 30 binds with the cohesin domain 21 of the scaffold protein 20 and self-organizes on the substrate 10.

Thus, the enzyme 30 can be arrayed on the substrate 10, which is made of carbon material and the like, through the dockerin domain 31. The temperature can be adjusted according to the enzyme 30 used.

According to the protein alignment method of this invention, for example, a partial structure of cellulosome can be produced by using a protein recombination technology, and by adding it to the graphite, multiple scaffold proteins 20 can be arrayed regularly along the hexagonal-plate-shaped crystals of the graphite. In addition, the enzyme subunits of cellulosome can be bonded to the scaffold proteins 20 arrayed on the graphite, causing their self-organization.

The technology for arraying proteins on the substrate 10 made of carbon materials such as graphite can be used for, for example, biochip, biosensor, biological device, microarray, chromatographic carrier, immobilized polymer, immobilized enzyme, taste sensor, prevention of nonspecific adsorption of protein, biochip for medical material, element for carrying protein drug, protein separation, and manufacture of functionalized molecule.

Specifically, the scaffold protein 20 is arrayed on the substrate 10 made of carbon material. Subsequently, by using, needless to say, the cellulosome enzyme subunit, which has the dockerin domain 31 as its original property, or proteins and the like, to which the dockerin domain 31 is artificially attached as its function by gene recombination, self-organization can be performed by the cohesin-dockerin interaction with the scaffold protein 20 arrayed on the substrate 10.

For the above proteins, hydrolytic enzyme, oxygen reduction enzyme, transfer enzyme, addition-elimination enzyme (lyase), isomerase, synthesis enzyme (ligase) and proteins, which have an enzyme function and EC number, such as those catalyzing epimerization reaction, intramolecular rearrangement and racemization reaction can be used. In addition, as protein, an antibody, labeled protein and chimera protein, in which a chemical agent is attached directly to the dockerin domain 31 by using conjugate, can be used according to the situation.

In addition to the above proteins, it is acceptable to use a low-molecular compound, labeled compound having radio isotope, and labeled compound having stable isotope.

The dockerin domain 31 can be directly labeled, or the dockerin domain 31, to which a compound having a labeling function is attached, can be used. Therefore, any form can be used as far as it has the dockerin domain 31 and can interact with the scaffold proteins 20 arrayed on the substrate 10 made of carbon and can align the proteins at least.

(Alternative of Protein Alignment Method)

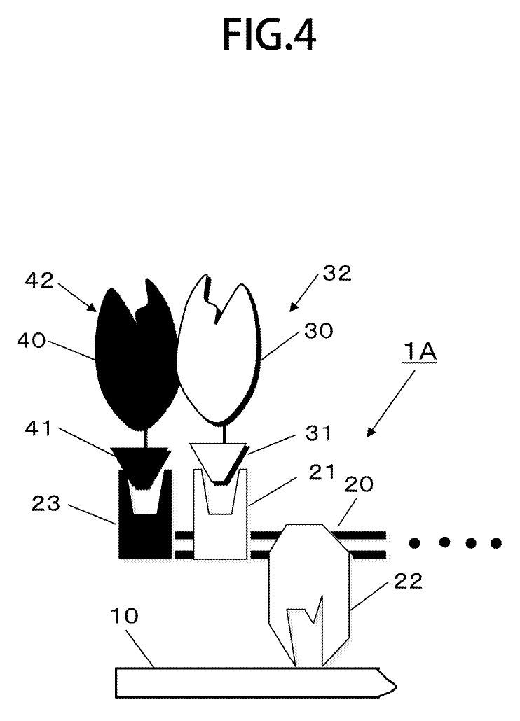

FIG. 4 shows a pattern diagram of the composite 1A related to an alternative of the protein alignment method of this invention. As shown in this figure, the composite 1A differs from the composite 1 shown in FIG. 1 in that the cohesin domain 23 of different microbial species is additionally bonded to the scaffold protein 20, which is bonded to the substrate 10 through the cellulose binding domain 22. Furthermore, the composite 1A differs from the composite 1 shown in FIG. 1 in that the functional protein 42 containing the dockerin is bonded to the cohesin domain 23 of different microbial species. The functional protein 42 containing this dockerin consists of the protein 40 of the same microbial species as that of the cohesin domain 23 and the dockerin domain 41.

It has been known that the above cohesin-dockerin interaction is specific to microbial species (see Non-Patent Literature 3). Namely, the dockerin domain 31 of Clostridium thermocellum binds with the cohesin domain 21 of Clostridium thermocellum, and the dockerin domain 41 of Clostridium cellulovorans binds with the cohesin domain 23 of Clostridium cellulovorans. Therefore, production of artificial cellulosome using these properties is reported (Non-Patent Literature 4).

By using this technology, when the cohesin domain 21 of the scaffold protein 20 bonded to the composite 1 in FIG. 1 is made to have the amino acid sequence of the cohesin domain 23 derived from different microbes and also the protein 40 such as enzyme is made to have the dockerin domain 33 of the microorganism derived from the cohesin domain 23, several types of different functional proteins 32 and 42 can be arranged (see Non-Patent Literature 4).

Thus, by making the cohesin domain 21 to have the amino acid sequence of the cohesin domain 23 derived from different microbial species, also making the protein 40 such as enzyme to have the dockerin domain 41 specific to its microbial species and using the microorganism-specific cohesin-dockerin interaction, different proteins 32 and 42 can be arranged on the scaffold protein 20.

For example, in the Non-Patent Literature 4, chimeric scaffold protein is produced by using the cohesin domain of Clostridium thermocellum, the cohesin domain of Clostridium thermocellum and the cohesin domain of R. flavefaciens. In addition, proteins can be arranged in an arbitrary place due to the cohesin-dockerin interaction species-specific to its chimeric scaffold protein by using the cellulase (family 9) derived from Clostridium thermocellum and having a dockerin domain, the cellulase (family 8) derived from C. cellulolyticum and having a dockerin domain, and the cellulase (family 9) derived from R. flavefaciens and having a dockerin domain. Furthermore, this invention is described in detail below by using examples. However, this invention is not limited to the following examples.

### Example

The model of the recombinant protein produced and used in this invention is shown in FIG. 1. The genome DNA from Clostridium thermocellum was extracted in the following procedure.

After Clostridium thermocellum was cultured in a BM7CO-CB culture medium containing 0.5% cellobiose, it was subjected to centrifugal separation at 4° C. and 10,000 revolutions for five minutes to collect the fungus body.

(Culturing of Clostridium thermocellum)

The Clostridium thermocellum JK-S14 strain (NITE BP-627) was cultured in a BM7CO-CB culture medium containing 10 g/L of fine crystal cellulose at 60° C. for four days.

Subsequently, to subject the obtained fungus body to bacteriolysis, 10% SDS (sodium lauryl sulfate) was added so that its final concentration became 0.5%, the proteinase K (1 mg/mL) solution was also added so that its final concentration became 5 μg/mL, and reaction was performed at 37° C. for one hour.

In addition, 10% cetyltrimethylammonium bromide—0.7 M (mole) sodium chloride solution was added so that its concentration became 1%, and reaction was performed at 65° C. for ten minutes. Subsequently, the equal amount of chloroform isoamyl alcohol solution was added, the solution was stirred well, and it was subjected to centrifugal separation of 15,000 revolutions for five minutes to obtain the water layer.

The equal amount of mixture of phenol, chloroform and isoamyl alcohol was added to the above water layer, and it was stirred and the subjected again to centrifugal separation of 15,000 revolutions for five minutes to obtain the water layer. Isopropanol 0.6 times its volume was added to this water layer and the genome DNA was separated out and the genome DNA was prepared by performing centrifugal separation again. This genome DNA was washed with 70% ethanol and dried.

The composition of the prepared BM7CO-CB culture medium was 1.5 g/L of potassium dihydrogen phosphate, 2.9 g/L of dibasic potassium phosphate, 2.1 g/L of urea, 6.0 g/L of yeast extract, 4 g/L of sodium carbonate, 0.05 g/L of cysteine hydrochloride, and 0.2 mL of mineral solution (MgCl₂/6H₂O 5 g, CaC₁₂/2H₂O 0.75 g and FeSO₄/6H₂O 0.0063 g were solved in 4 mL of water). In addition, cellobiose was added as a carbon source to the culture medium so that its final concentration became 5 g/L. The final culture medium pH was adjusted around 7.0.

Using the above prepared genome DNA, CipA was designed and synthesized from the oligonucleotide primer CipA (SEQUENCE No. 1: AGTCA GAGCTCC GCC ACA ATG ACA GTCGAG) and CipAR (SEQUENCE No. 2: AGTCACTCGAGTACAGGCTGTGTTGATGG), mCipA was designed and synthesized from the oligonucleotide primer mCipAF (SEQUENCE No. 3: AGTCA GAGCTCC GCC ACA ATG ACA GTCGAG) and mCipAR (SEQUENCE No. 4: AGTCA CTCGAG TACAGGCTGTGTTGATGG), CelA was designed and synthesized from oligonucleotide primer CelAF (SEQUENCE No. 5: AGTCA GAGCTCC GCA GGT GTG CCT TTT AAC) and CelAR (SEQUENCE No. 6: AGTC AAGCTT ATAAGGTAGGTGGGGTATG-3), and CelS was designed and synthesized from oligonucleotide primer CelSA (SEQUENCE No. 7: AGTC GAGCTCC GGT CCT ACA AAG GCACCTA) and CelSR (SEQUENCE No. 8: AGTCA GCTGAGC GTTCTTGTACGGCAATGT). In PCR, which is a method for amplifying genes, about 5.5 kilobase long double-strand amplified DNA sequence was obtained. The amplified CipA gene sequence is shown in the SEQUENCE No. 9.

The designed oligonucleotide primers CipF and CipAR are attached with the restriction enzyme site Sacl and the Xhol site to insert them into E coli expression vector.

Similarly, oligonucleotide primer mCipAR is attached with the restriction enzyme site Xhol site. Oligonucleotide primer CelAF, CelAR. CelS, oligonucleotide primer CelSA and CelSR are attached with restriction enzyme site Sacl and Hind III, SacI and Bpu11021.

CipA gene sequence, which is the scaffold protein 20 of Clostridium thermocellum, CelA gene sequence and CelS gene sequence can be obtained through the website (http://www.Ncbi.nlm.nih.gov/) of the National Center for Biotechnology Information (NBIC)(CipA GenBank accession No.: CAA91220.1), (CelA GenBank accession No.: K03088), and (CelS GenBank accession No. L06942.1).

In PCR, 16srRNA gene was amplified by the ExTaq DNA polymerase (Takara BIO Inc.) The PCR condition was thirty cycles of 98° C. for one minute, 55° C. for one minute and 72° C. for four minutes, and amplification was performed in this condition.

After the amplified band was checked by 0.8% agarose gel electrophoresis, the PCR products were refined by the QIAGEN PCR refining kit (QIAGEN GmbH). Using the above restriction enzyme (Takara BIO Inc.), the refined PCR products were subjected to restriction enzyme treatment at 37° C. overnight.

The restriction-enzyme-treated PCR product was subjected to the 0.8% agarose gel electrophoresis again to separate the restriction enzyme decomposition product, and the intended band was cut from the gel and refined by the gel extraction kit (QIAGEN GmbH).

The pET22b expression vector (Merck KGaA) was used to express the CipA gene in E coli. This vector was so designed that 6-residue histidine tag was fused to the N-terminal side of the intended protein to be expressed. The pET19b (Merck KGaA) expression vector was used for expressing CelS in E coli. In this case, restriction enzyme treatment was performed at 37° C. overnight using Sacl and Bpu1102. After the restriction enzyme treatment was performed, further treatment with Alkaline Phosphatase (Takara BIO Inc.) at 50° C. for one hour was performed to dephosphorylate the restriction enzyme cutting site. After extraction of phenol chloroform was repeated and Alkaline Phosphatase was deactivated, ethanol precipitation treatment was performed, and the restriction-enzyme-treated pET19b expression vector was collected.

To construct CipA expression vector, mCipA expression vector, CelA expression vector and CelS expression vector, the above restriction-enzyme-treated pET22b and pET19b expression vectors were incubated with the T4 ligase (Takara BIO Inc.) at 16° C. overnight and their fragments were connected.

Subsequently, the above expression vectors CipA-pET22b, mCipA-pET22b, CelA-pET22b and CelS-pET19b were transformed into E coli JM109 and cultured at 37° C. overnight in a Luria-Bertani culture medium (called LB culture medium) containing 50 μg/mL of ampicillin sodium and 1.5% agar.

The composition of the LB culture medium is shown below.

### Bacto Peptone 1 g/L

Sodium chloride 1 g/L
Yeast extract 1 g/L
The final culture medium pH was adjusted to around 7.0.

The clone having the intended gene fragment was selected from the grown colony. For the selection, the expression vectors were extracted from the E coli clone using the plasmid extraction kit (QIAGEN GmbH). After extracting the expression vectors, with the above primer, the DNA sequence was analyzed by BigDye (registered trademark) Terminator v3.1 (Applied Biosystems), PRISM (registered trademark) 3100 Genetic Analyzer (Applied Biosystems) or PRISM (registered trademark) 3700 DNA Analyzer (Applied Biosystems).

To check whether the gene sequence was correct, homology search was performed on the website of the National Center for Biotechnology Information (NBIC) using the obtained DNA sequence data. To express a large quantity of respective proteins, the expression vectors having the correct gene sequence were transformed into the E coli BL21 (Merck KGaA) again and the clones of E coli expressing a large quantity of protein were obtained.

To obtain CipA, mCipA, CelA and CelS, the E coli BL21 having respective expression vectors were cultured at 37° C. for four hours in a LB culture medium containing ampicillin sodium. After the culturing process, isopropyl-D-thiogalactopyranoside was added in concentration of 1 mM and further cultured for twelve hours.

Respective E coli BL 21 (DE3) was subjected to centrifugal separation (8,000 revolutions, 4° C. and 10 minutes), and the E coli was collected. The collected E coli was frozen at −80° C. overnight, suspended in the bacteriolysis buffer solution (50 mM of phosphate buffer solution, 300 mM of sodium chloride, 10 mM of imidazole, and pH 8.0) and then shattered in ice by a sonicator. After the obtained bacteriolysis suspension was subjected to centrifugal separation to separate the transparent lysis solution from the precipitated unshattered fungus body, only the transparent lysis solution was collected and filtered by a 0.45 μm filter.

The His-tag fused protein was obtained from the lysis solution through the nickel agarose gel column (Ni-NTA agarose gel QIAGEN GmbH). In addition, the eluted recombinant protein was refined through the desalting column (Bio-Rad Laboratories, Inc.) After refined proteins were diluted with distilled water as needed, amounts of respective protein is measured by the BCA-protein measuring kit (Thermo Scientific). The analytical curve of protein was prepared by using the bovine serum albumin.

The CipA gene sequence and its amino-acid sequence are shown in the SEQUENCE No. 9 and 10, respectively.

The gene sequence and the amino-acid sequence of mCipA, which is a partial structure of CipA of Clostridium thermocellum, are shown in the SEQUENCE No. 11 and 12, respectively.

The gene sequence and the amino-acid sequence of the typical cohesin domain 21, which CipA of Clostridium thermocellum has, are shown in the SEQUENCE No. 13 and 14, respectively.

The gene sequence, which codes the amino-acid sequence of the cellulose binding domain 22 (CBM) of Clostridium thermocellum, and the gene sequence of the cellulose binding domain 22 (CBM) of Clostridium thermocellum are shown in the SEQUENCE No. 15 and 16, respectively.

The gene sequence and amino-acid sequence of CelA are shown in the SEQUENCE No. 18 and 19.

The gene sequence and amino-acid sequence of CelS are shown in the SEQUENCE No. 20 and 21, respectively.

The gene sequence and amino-acid sequence of dockerin domain 31 of CelA are shown in the SEQUENCE No. 21 and 22, respectively.

The gene sequence and amino-acid sequence of dockerin domain 31 of CelS are shown in the SEQUENCE No. 23 and 24, respectively.

FIG. 1 shows the protein domain structure in a pattern diagram based on the above amino-acid sequence. Observation was conducted by a high-speed atomic force microscope developed by Kodera at al. (see the Non-Patent Literature 2).

For observation by a high-speed atomic force microscope, the BL-AC10EGS-A2 small cantilever (Olympus Corporation) and the Nano Live Version (Olympus Corporation) were used.

For the graphite used as a base, the highly oriented pyrolytic graphite (called HOPG) was used. The less than 0.1 mm thick HOPG was attached to a cylindrical glass stage (2 mm high and 1.5 mm diameter), and a Scotch tape was pressed to ensure the flatness of the HOPG surface.

The respective proteins of highly refined CipA, mCipA, CelA and CelS were prepared by 50 mM of acetic acid buffer solution (pH 6.0) to 0.2 μM to 20 μM to use them for the above high-speed atomic force microscope.

About 2 μM of the above CipA and mCipA were added on the HOPG, and one to four frames of high-speed atomic force microscope image were taken within one second. Analysis of the high-speed atomic force microscope images was conducted by using the IGOR pro (WaveMetrics). All the high-speed atomic force microscope images were treated by a 2×2 pixel averaging filter to reduce noise spikes in the high-speed atomic force microscope.

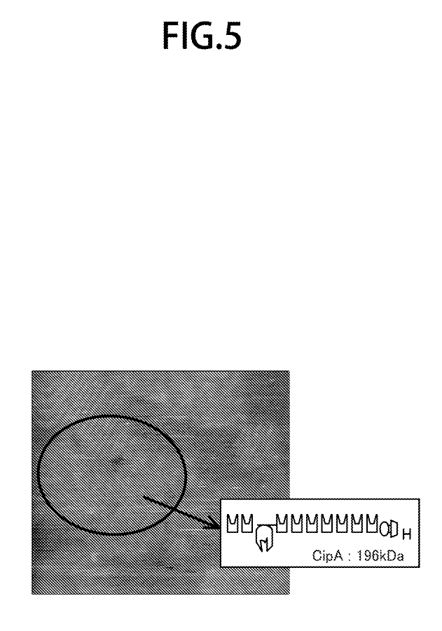

FIG. 5 shows a high-speed atomic force microscope image taken when CipA, which is the scaffold protein 20, was arrayed on the HOPG sample disk. As clearly shown by this figure, CipA was arrayed along the hexagonal carbon grid of the HOPG disk.

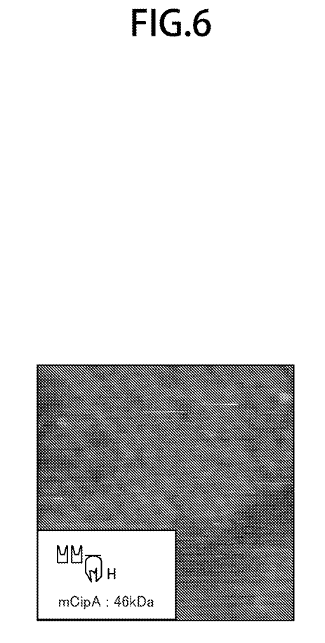

mCipA was also added to the HOPG sample disk same as CipA was done to check whether even a partial domain of CipA would be arrayed. FIG. 6 shows a high-speed atomic force microscope image taken when mCipA was arranged on the HOPG sample disk. As clearly shown by this figure, mCipA was arrayed along the hexagonal carbon grid of the HOPG disk same as CipA shown in FIG. 5.

(Bonding of Scaffold Protein and Functional Protein)

Usually, when non-specific adsorption occurs to protein, a structural change is caused and protein loses its function. If CipA was adsorbed non-specifically to HOPG, it was expected that the scaffold protein 20 would lose its function. Two enzyme subunits CelA and CelS were prepared and a bonding test was conducted to confirm whether the enzyme subunit of cellulosome could bind with CipA arranged on the HOPG and could perform self organization.

As similar to described before, 2 μL of CelA and CelS were added on the HOPG sample disk shown before, where mCipA was arrayed, and one to four frames of high-speed atomic force microscope image were taken within one second. Analysis of the high-speed atomic force microscope images was conducted by using the IGOR pro (WaveMetrics) same as the above.

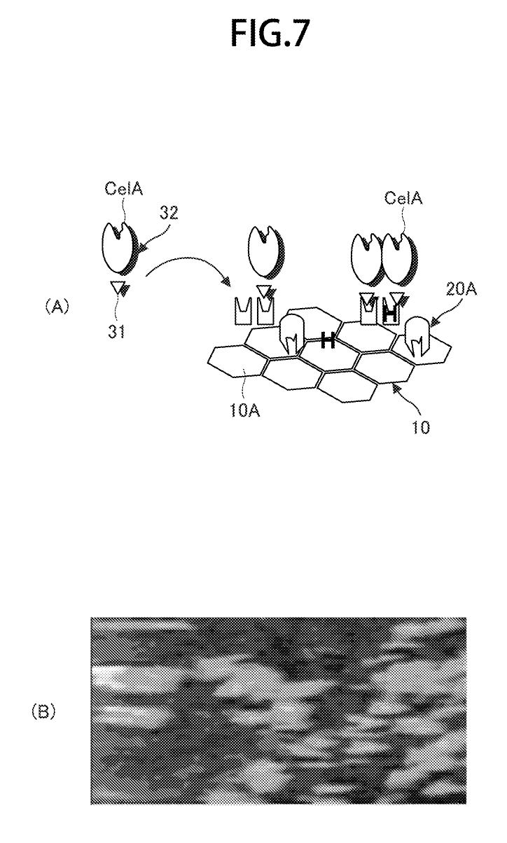

FIG. 7 shows the bond between the scaffold protein 20 and CelA. (a) shows a pattern diagram of the bond, and (b) shows a high-speed atomic force microscope image.

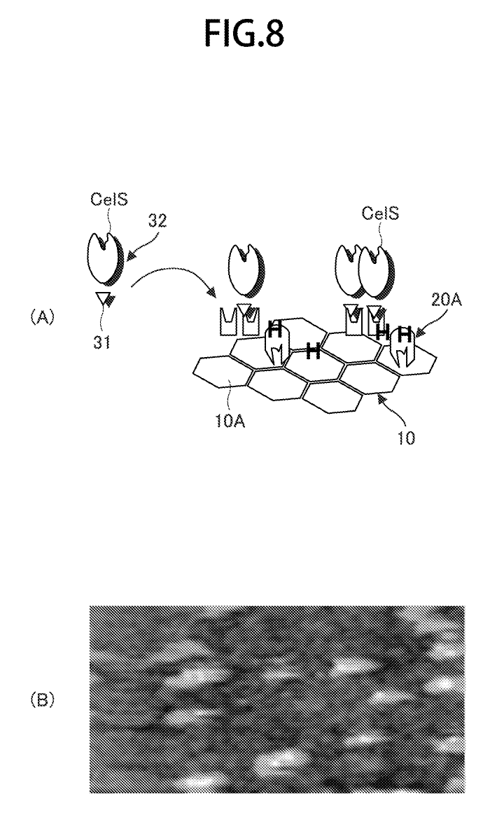

FIG. 8 shows the bond between the scaffold protein 20 and CelS; (a) shows a pattern diagram of the bond, and (b) shows a high-speed atomic force microscope image.

What are bright in yellow in the high-speed atomic force microscope images of FIGS. 7 and 8 are CelA and CelS enzyme proteins, and it was shown that they were interacting with mCipA, which was the partial scaffold protein 20A arrayed regularly on the HOPG sample disk.

Based on the results shown in above FIGS. 7 and 8, it was understood that non-specific adsorption did not occur in the scaffold protein 20 of CipA, that the protein was arrayed regularly while keeping its structure without losing its function as a protein, and that the cohesin domain 21 of the scaffold protein (20) interacted with the dockerin domain 31 possessed by the protein, self-organizing a cellulosome structure.

Therefore, by using the above method, not only enzymes but also desired proteins such as the functional protein 32, which contains a antibody and dockerin, can be arrayed on the surface of substrate 10 without losing their function.

This invention is not limited to the above examples, but can be used in various different forms within the scope of the invention indicated in the patent claim, and it is needless to say that such usage is also included within the scope of this invention.

Accession Number

NITE BP-627 (Clostridium thermocellum JK-S14 strain)

Depository Designation

Name of deposit institution: NPMD National Institute of Technology and Evaluation NITE Patent Microorganisms Depositary (NPMD)

Address of deposit institution: 2-5-8 Kazusa-Kamatari, Kisarazu, Chiba, 292-0818 Japan
Deposition date: Aug. 12, 2008
Deposit No. NPMD NITE BP627

1. A method for aligning proteins, comprising; arraying proteins on a carbon substrate.

2. The method for aligning proteins according to claim 1, wherein a cellulose binding domain, a cohesin domain, and a dockerin domain derived from cellulosome are used for aligning the proteins.

3. The method for aligning proteins according to claim 2, comprising arraying a structure consisting of the cellulose binding domain of cellulosome and the cohesin domains, and combining a protein having the dockerin domain with the cohesin domain.

4. The method for aligning proteins according to claim 2, wherein the cohesin domains have amino-acid sequences of cohesin domains derived from several different microbial species, protein such as enzymes are made to have a dockerin domain, which the microbial species have, and the cohesin-dockerin interaction specific to the microbial species is used to arrange several types of protein.

5. The method for aligning proteins according to claim 1, wherein the substrate is made of a mineral material including carbon.

6. The method for aligning proteins according to claim 5, wherein the substrate is made of graphite.

7. A composite substrate made of carbon, on which the proteins are arrayed by one of the protein alignment methods according to claim 1.

8. A composite substrate made of carbon, on which the proteins are arrayed by one of the protein alignment methods according to claim 2.

9. A composite substrate made of carbon, on which the proteins are arrayed by one of the protein alignment methods according to claim 3.

10. A composite substrate made of carbon, on which the proteins are arrayed by one of the protein alignment methods according to claim 4.

11. A composite substrate made of carbon, on which the proteins are arrayed by one of the protein alignment methods according to claim 5.

12. A composite substrate made of carbon, on which the proteins are arrayed by one of the protein alignment methods according to claim 6.


SEQUENCE LISTING (24 sequences)

SEQ ID NO: 1 - DNA, 30 bp, Clostridium thermocellum
agtcagagctccgccacaatgacagtcgag

SEQ ID NO: 2 - DNA, 29 bp, Clostridium thermocellum
agtcactcgagtacaggctgtgttgatgg

SEQ ID NO: 3 - DNA, 30 bp, Clostridium thermocellum; identical to SEQ ID NO: 1

SEQ ID NO: 4 - DNA, 29 bp, Clostridium thermocellum
agtcactcgagtacaggctgtgttgatgg

SEQ ID NO: 5 - DNA, 30 bp, Clostridium thermocellum
agtcagagctccgcaggtgtgccttttaac

SEQ ID NO: 6 - DNA, 29 bp, Clostridium thermocellum
agtcaagcttataaggtaggtggggtatg

SEQ ID NO: 7 - DNA, 30 bp, Clostridium thermocellum
agtcgagctccggtcctacaaaggcaccta

SEQ ID NO: 8 - DNA, 30 bp, Clostridium thermocellum
agtcagctgagcgttcttgtacggcaatgt

SEQ ID NO: 9 - DNA, 5573 bp, Clostridium thermocellum
atgagaaaagtcatcagtatgctcttagttgtggctatgctgacgacgatttttgcggcgatgataccgcagacagtatcggcggccacaatgacagtcgagatcggcaaagttacagcagccgttggatcaaaagtagaaatacctataaccctgaaaggagtgccatccaaaggaatggccaattgcgacttcgtattgggttatgatccaaatgtgctggaagtaacagaagtaaaaccaggaagcataataaaagatccggatcctagcaagagctttgatagcgcaatatatccggatcgaaagatgattgtatttctgtttgcagaagacagtggaagaggaacgtatgcaataactcaggatggagtatttgcaacaattgtagccactgtcaaatcagctgcagcggcaccgattactttgcttgaagtaggtgcatttgcggacaacgatttagtagaaataagcacaacttttgtcgcgggcggagtaaatcttggtagttccgtaccgacaacacagccaaatgttccgtcagacggtgtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgttggcaatgcaacaccgaccaagggagcaacaccaacaaatacagctacgccgacaaaatcagctacggctacgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagatgattcgaatgcagtaaggattaaggtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagaattgatagttgacccgaatcctaccaagagctttgatactgcagtatatcctgacagaaagatgatagtattcctgtttgcggaagacagcggaacaggagcgtatgcaataactgaagatggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacgggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagtacctacaacatcgccgacaacaacaccgccagagccgacgataactccgaacaagttgacacttaagataggcagagcagaaggaagacctggagacacggtggaaataccggttaacttgtatggagtacctcaaaaaggaatagcaagcggtgacttcgtagtaagctatgacccgaatgtacttgagataatagagatagaaccgggagaattgatagttgacccgaatcctaccaagagctttgatactgctgcagtatatcctgacagaaagatgatagtattcctgtttgcggaagacagcggaacaggagcgtatgcaataactgaagatggagtatttgctacgatagtagcgaaagtaaaagaaggagcacctgaaggattcagtgcaatagaaatttctgagtttggtgcatttgcagataatgatctggtagaagtggaaactgaccttatcaatggtggagtacttgtaactaataaacctgtaatagaaggatataaagtatccggatacattttgccagacttctccttcgacgctactgttgcaccacttgtaaaggccggattcaaagttgaaatagtaggaacagaattgtatgcagtaacagatgcaaacggatactttgaaataaccggagtacctgcaaatgcaagcggatatacattgaagatttcaagagcaacttacttggacagagtaattgcaaatgttgtagtaacgggagatacttcagtttcaacttcacaggctccaataatgatgtgggtaggagacatagtgaaagacaattctatcaacctgttggacgttgcagaagttatccgttgcttcaacgctactaaaggaagcgcaaactacgtagaagaacttgacattaatagaaacggcgcaattaacatgcaagacataatgattgttcataagcactttggagctacatcaagtgattacgacgcacagtaaatattaaa

SEQ ID NO: 10 - protein, 1854 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGELIVDPNPTKSFDTAVYPDRKMIVFLFAEDSGTGAYAITEDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTVPTTSPTTTPPEPTITPNKLTLKIGRAEGRPGDTVEIPVNLYGVPQKGIASGDFVVSYDPNVLEIIEIEPGELIVDPNPTKSFDTAAVYPDRKMIVFLFAEDSGTGAYAITEDGVFATIVAKVKEGAPEGFSAIEISEFGAFADNDLVEVETDLINGGVLVTNKPVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDYDAQ

SEQ ID NO: 11 - DNA, 1599 bp, Clostridium thermocellum
atgagaaaagtcatcagtatgctcttagttgtggctatgctgacgacgatttttgcggcgatgataccgcagacagtatcggcggccacaatgacagtcgagatcggcaaagttacagcagccgttggatcaaaagtagaaatacctataaccctgaaaggagtgccatccaaaggaatggccaattgcgacttcgtattgggttatgatccaaatgtgctggaagtaacagaagtaaaaccaggaagcataataaaagatccggatcctagcaagagctttgatagcgcaatatatccggatcgaaagatgattgtatttctgtttgcagaagacagtggaagaggaacgtatgcaataactcaggatggagtatttgcaacaattgtagccactgtcaaatcagctgcagcggcaccgattactttgcttgaagtaggtgcatttgcggacaacgatttagtagaaataagcacaacttttgtcgcgggcggagtaaatcttggtagttccgtaccgacaacacagccaaatgttccgtcagacggtgtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgttggcaatgcaacaccgaccaagggagcaacaccaacaaatacagctacgccgacaaaatcagctacggctacgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgta

SEQ ID NO: 12 - protein, 533 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPV

SEQ ID NO: 13 - DNA, 408 bp, Clostridium thermocellum
atgacagtcgagatcggcaaagttacagcagccgttggatcaaaagtagaaatacctataaccctgaaaggagtgccatccaaaggaatggccaattgcgacttcgtattgggttatgatccaaatgtgctggaagtaacagaagtaaaaccaggaagcataataaaagatccggatcctagcaagagctttgatagcgcaatatatccggatcgaaagatgattgtatttctgtttgcagaagacagtggaagaggaacgtatgcaataactcaggatggagtatttgcaacaattgtagccactgtcaaatcagctgcagcggcaccgattactttgcttgaagtaggtgcatttgcggacaacgatttagtagaaataagcacaacttttgtcgcgggcggagta

SEQ ID NO: 14 - protein, 136 aa, Clostridium thermocellum
MTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGV

SEQ ID NO: 15 - protein, 237 aa, Clostridium thermocellum
ATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPN

SEQ ID NO: 16 - DNA, 780 bp, Clostridium thermocellum
ggatttgcagataatgacctggtagaacagaaggtatcatttatagacggtggtgttaacgttggcaatgcaacaccgaccaagggagcaacaccaacaaatacagctacgccgacaaaatcagctacggctacgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaat

SEQ ID NO: 17 - DNA, 1434 bp, Clostridium thermocellum
gtgaagaacgtaaaaaaaagagtaggtgtggttttgctgattcttgcagtgttgggggtttatatgttggcaatgccggcaaacactgtgtcagcggcaggtgtgccttttaacacaaaatacccctatggtcctacttctattgccgataatcagtcggaagtaactgcaatgctcaaagcagaatgggaagactggaagagcaagagaattacctcgaacggtgcaggaggatacaagagagtacagcgtgatgcttccaccaattatgatacggtatccgaaggtatgggatacggacttcttttggcggtttgctttaacgaacaggctttgtttgacgatttataccgttacgtaaaatctcatttcaatggaaacggacttatgcactggcacattgatgccaacaacaatgttacaagtcatgacggcggcgacggtgcggcaaccgatgctgatgaggatattgcacttgcgctcatatttgcggacaagttatggggttcttccggtgcaataaactacgggcaggaagcaaggacattgataaacaatctttacaaccattgtgtagagcatggatcctatgtattaaagcccggtgacagatggggaggttcatcagtaacaaacccgtcatattttgcgcctgcatggtacaaagtgtatgctcaatatacaggagacacaagatggaatcaagtggcggacaagtgttaccaaattgttgaagaagttaagaaatacaacaacggaaccggccttgttcctgactggtgtactgcaagcggaactccggcaagcggtcagagttacgactacaaatatgatgctacacgttacggctggagaactgccgtggactattcatggtttggtgaccagagagcaaaggcaaactgcgatatgctgaccaaattctttgccagagacggggcaaaaggaatcgttgacggatacacaattcaaggttcaaaaattagcaacaatcacaacgcatcatttataggacctgttgcggcagcaagtatgacaggttacgatttgaactttgcaaaggaactttatagggagactgttgctgtaaaggacagtgaatattacggatattacggaaacagcttgagactgctcactttgttgtacataacaggaaacttcccgaatcctttgagtgacctttccggccaaccgacaccaccgtcgaatccgacaccttcattgcctcctcaggttgtttacggtgatgtaaatggcgacggtaatgttaactccactgatttgactatgttaaaaagatatctgctgaagagtgttaccaatataaacagagaggctgcagacgttaatcgtgacggtgcgattaactcctctgacatgactatattaaagagatatctgataaagagcataccccacctaccttattag

SEQ ID NO: 18 - protein, 477 aa, Clostridium thermocellum
MKNVKKRVGVVLLILAVLGVYMLAMPANTVSAAGVPFNTKYPYGPTSIADNQSEVTAMLKAEWEDWKSKRITSNGAGGYKRVQRDASTNYDTVSEGMGYGLLLAVCFNEQALFDDLYRYVKSHFNGNGLMHWHIDANNNVTSHDGGDGAATDADEDIALALIFADKLWGSSGAINYGQEARTLINNLYNHCVEHGSYVLKPGDRWGGSSVTNPSYFAPAWYKVYAQYTGDTRWNQVADKCYQIVEEVKKYNNGTGLVPDWCTASGTPASGQSYDYKYDATRYGWRTAVDYSWFGDQRAKANCDMLTKFFARDGAKGIVDGYTIQGSKISNNHNASFIGPVAAASMTGYDLNFAKELYRETVAVKDSEYYGYYGNSLRLLTLLYITGNFPNPLSDLSGQPTPPSNPTPSLPPQVVYGDVNGDGNVNSTDLTMLKRYLLKSVTNINREAADVNRDGAINSSDMTILKRYLIKSIPHLPY

SEQ ID NO: 19 - DNA, 2226 bp, Clostridium thermocellum
atggtaaaaagcagaaagatttctattctgttggcagttgcaatgctggtatccataatgatacccacaactgcattcgcaggtcctacaaaggcacctacaaaagatgggacatcttataaggatcttttccttgaactctacggaaaaattaaagatcctaagaacggatatttcagcccagacgagggaattccttatcactcaattgaaacattgatcgttgaagcgccggactacggtcacgttactaccagtgaggctttcagctattatgtatggcttgaagcaatgtatggaaatctcacaggcaactggtccggagtagaaacagcatggaaagttatggaggattggataattcctgacagcacagagcagccgggtatgtcttcttacaatccaaacagccctgccacatatgctgacgaatatgaggatccttcatactatccttcagagttgaagtttgataccgtaagagttggatccgaccctgtacacaacgaccttgtatccgcatacggtcctaacatgtacctcatgcactggttgatggacgttgacaactggtacggttttggtacaggaacacgggcaacattcataaacaccttccaaagaggtgaacaggaatccacatgggaaaccattcctcatccgtcaatagaagagttcaaatacggcggaccgaacggattccttgatttgtttacaaaggacagatcatatgcaaaacagtggcgttatacaaacgctcctgacgcagaaggccgtgctatacaggctgtttactgggcaaacaaatgggcaaaggagcagggtaaaggttctgccgttgcttccgttgtatccaaggctgcaaagatgggtgacttcttgagaaacgacatgttcgacaaatacttcatgaagatcggtgcacaggacaagactcctgctaccggttatgacagtgcacactaccttatggcctggtatactgcatggggtggtggaattggtgcatcctgggcatggaagatcggatgcagccacgcacacttcggatatcagaacccattccagggatgggtaagtgcaacacagagcgactttgctcctaaatcatccaacggtaagagagactggacaacaagctacaagagacagcttgaattctatcagtggttgcagtcggctgaaggtggtattgccggtggagcaaccaactcctggaacggtagatatgagaaatatcctgctggtacgtcaacgttctatggtatggcatatgttccgcatcctgtatacgctgacccgggtagtaaccagtggttcggattccaggcatggtcaatgcagcgtgtaatggagtactacctcgaaacaggagattcatcagttaagaatttgattaagaagtgggtcgactgggtaatgagcgaaattaagctctatgacgatggaacatttgcaattcctagcgacctcgagtggtcaggtcagcctgatacatggaccggaacatacacaggcaacccgaacctccatgtaagagtaacttcttacggtactgaccttggtgttgcaggttcacttgcaaatgctcttgcaacttatgccgcagctacagaaagatgggaaggaaaacttgatacaaaagcaagagacatggctgctgaactggttaaccgtgcatggtacaacttctactgctctgaaggaaaaggtgttgttactgaggaagcacgtgctgactacaaacgtttctttgagcaggaagtatacgttccggcaggttggagcggtactatgccgaacggtgacaagattcagcctggtattaagttcatagacatccgtacaaaatatagacaagatccttactacgatatagtatatcaggcatacttgagaggcgaagctcctgtattgaattatcaccgcttctggcatgaagttgaccttgcagttgcaatgggtgtattggctacatacttcccggatatgacatataaagtacctggtactccttctactaaattatacggcgacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctcaaagaaatagatacattgccgtacaagaactaa

SEQ ID NO: 20 - protein, 741 aa, Clostridium thermocellum
MVKSRKISILLAVAMLVSIMIPTTAFAGPTKAPTKDGTSYKDLFLELYGKIKDPKNGYFSPDEGIPYHSIETLIVEAPDYGHVTTSEAFSYYVWLEAMYGNLTGNWSGVETAWKVMEDWIIPDSTEQPGMSSYNPNSPATYADEYEDPSYYPSELKFDTVRVGSDPVHNDLVSAYGPNMYLMHWLMDVDNWYGFGTGTRATFINTFQRGEQESTWETIPHPSIEEFKYGGPNGFLDLFTKDRSYAKQWRYTNAPDAEGRAIQAVYWANKWAKEQGKGSAVASVVSKAAKMGDFLRNDMFDKYFMKIGAQDKTPATGYDSAHYLMAWYTAWGGGIGASWAWKIGCSHAHFGYQNPFQGWVSATQSDFAPKSSNGKRDWTTSYKRQLEFYQWLQSAEGGIAGGATNSWNGRYEKYPAGTSTFYGMAYVPHPVYADPGSNQWFGFQAWSMQRVMEYYLETGDSSVKNLIKKWVDWVMSEIKLYDDGTFAIPSDLEWSGQPDTWTGTYTGNPNLHVRVTSYGTDLGVAGSLANALATYAAATERWEGKLDTKARDMAAELVNRAWYNFYCSEGKGVVTEEARADYKRFFEQEVYVPAGWSGTMPNGDKIQPGIKFIDIRTKYRQDPYYDIVYQAYLRGEAPVLNYHRFWHEVDLAVAMGVLATYFPDMTYKVPGTPSTKLYGDVNDDGKVNSTDAVALKRYVLRSGISINTDNADLNEDGRVNSTDLGILKRYILKEIDTLPYKN

SEQ ID NO: 21 - DNA, 60 bp, Clostridium thermocellum
gacgttaatcgtgacggtgcgattaactcctctgacatgactatattaaagagatatctg

SEQ ID NO: 22 - protein, 20 aa, Clostridium thermocellum
DVNRDGAINSSDMTILKRYL

SEQ ID NO: 23 - DNA, 159 bp, Clostridium thermocellum
gacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctc

SEQ ID NO: 24 - protein, 53 aa, Clostridium thermocellum
DVNDDGKVNSTDAVALKRYVLRSGISINTDNADLNEDGRVNSTDLGILKRYIL